(19) United States
(12) Patent Application Publication
(10) Pub. No.: US 2018/0344538 A1
(43) Pub. Date: Dec. 6, 2018

(54) ABSORBENT ARTICLE

(71) Applicant: Kao Corporation, Tokyo (JP)

(72) Inventors: Takeshi SUZUKI, Shimotsuke-shi, Tochigi (JP); Mina TOMITA, Utsunomiya-shi, Tochigi (JP); Akihito AKUTAGAWA, Saitama-shi, Saitama (JP); Ayuka MINAMIZAKI, Utsunomiya-shi, Tochigi (JP)

(73) Assignee: Kao Corporation, Tokyo (JP)

(21) Appl. No.: 15/780,553

(22) PCT Filed: Oct. 27, 2016
(86) PCT No.: PCT/JP2016/081950

§ 371 Date: May 31, 2018

(30) Foreign Application Priority Data
Dec. 1, 2015 (JP) 2015-234411
Oct. 12, 2016 (JP) 2016-201197

(51) Int. Cl. A61F 13/511; A61F 13/531

(52) U.S. Cl. CPC A61F 13/51104; A61F 13/513; A61F 13/531; A61F 13/5116

(57) ABSTRACT

An absorbent article including an absorbent member and a topsheet 2 on the skin facing side of the absorbent member is disclosed. The topsheet 2 includes a stack of a first sheet 20 and a second sheet 21. The topsheet 2 has a plurality of projections 23 resulting from the first sheet 20 protruding toward the skin of a wearer. The topsheet satisfies the following conditions: (1) the projections have a height of 0.5 to 10 mm; (2) the bonds 22 have a density of 10 to 30 bonds per a randomly chosen rectangular region measuring 10 mm in length and 15 mm in width in plan view; (3) the bonds in the rectangular region have a total area ratio of 12% to 35% relative to the area of the rectangular region; and (4) the topsheet has a shear strength of 21 to 70 N.

### TECHNICAL FIELD

The present invention relates to absorbent articles, such as disposable diapers and sanitary napkins.

### BACKGROUND ART

An absorbent article generally includes an absorbent member containing pulp and an absorbent polymer and a topsheet disposed on the skin facing side of the absorbent member. The topsheet, being a member with a larger skin contact area than any other members constituting the absorbent article, is one of the factors having a potent influence on wearer comfort. For use in absorbent articles, there is known a topsheet having an embossed or otherwise textured surface on its skin facing side, such a surface being formed by pressing process such as embossing. Also known is a technique for texturing the skin facing side of a topsheet to form spaces between the sheet and the wearer's skin, thereby dissipating humidity generated from discharged urine or menstrual blood. For example, Patent Literature 1 below discloses a three-dimensionally textured sheet composed of a first nonwoven layer and a second nonwoven layer fusion-bonded to each other in parts to form bonds. The first nonwoven layer protrudes away from the second nonwoven layer in non-bonded regions each surrounded by the bonds to form a large number of hollow projections. Patent Literature 1 mentions that the height of the projections preferably ranges from 1 to 10 mm with a view to improving fluid leakage resistance.

Patent Literature 2 below discloses an absorbent article including a hydrophobic porous topsheet, liquid impermeable backsheet, and an absorbent member interposed between the two sheets and additionally having a hydrophilic sheet between the porous topsheet and the absorbent member. The porous topsheet and the hydrophilic sheet are bonded to each other at a plurality of debosses formed by embossing over a part of or the entire area of the sheets. According to the drawings of Patent Literature 2, the debosses have a circular plan-view shape and are arranged in a dot pattern. Patent Literature 2 mentions that the debossed area ratio in the region of the article adapted to face the wearer's point of fluid discharge is preferably 7% or higher in view of improving the rate of absorbing body fluids, such as urine.

### CITATION LIST

### Patent Literature

Patent Literature 1: JP 2004-174234A

Patent Literature 2: JP 2004-49697A

### SUMMARY OF INVENTION

A three-dimensionally textured topsheet of the type described in Patent Literature 1, i.e., a composite of two nonwoven layers bonded together at a plurality of bonds with projections formed at non-bonded portions is advantageous in terms of reduced friction with the wearer's skin and good hand. Yet, further improvements on these characteristics are still needed in the present situation. When the shape or the like of projections are altered variously in an attempt to meet the needs of improving feel to the tough or hand, it results in changes of friction between the projections and the wearer's skin. Consequently, separation of the two nonwoven layers from each other tends to occur, starting from the bonds and, as a result, the skin contact side nonwoven layer of the topsheet can peel, lift off, and cling to the skin. This may result in reduction of wearer comfort. A topsheet satisfying both the requirements of softness and prevention of lift-off has not yet been provided.

The present invention provides an absorbent article including an absorbent member and a topsheet on the skin facing side of the absorbent member and having a longitudinal direction corresponding to the front-to-back direction of a wearer and a lateral direction perpendicular to the longitudinal direction. The topsheet includes a stack of a first sheet and a second sheet that are bonded to each other at a plurality of bonds. The first sheet has a plurality of projections toward the skin of a wearer resulting from the first sheet protruding away from the second sheet at portions other than the bonds. The topsheet satisfies the following conditions:

(1) the projections have a height of 0.5 to 10 mm;
(2) the bonds have a density of 10 to 30 bonds per a randomly chosen rectangular region of the topsheet measuring 10 mm in length and 15 mm in width in plan view;
(3) the bonds in the rectangular region have a total area ratio of 12% to 35% relative to the area of the rectangular region; and
(4) the topsheet has a shear strength of 21 to 70 N.

### BRIEF DESCRIPTION OF DRAWINGS

FIG. 1 is a plan view schematically illustrating the skin facing side, i.e., the topsheet side, of a disposable diaper as an embodiment of the absorbent article according to the invention in its flat-out configuration with every elastic member uncontracted.

FIG. 2 is a schematic cross-sectional view taken along line I-I of FIG. 1.

FIG. 3 is a fragmentary perspective view schematically showing the skin facing side of the topsheet used in the diaper of FIG. 1.

FIG. 4 is a fragmentary plan view schematically showing the skin facing side of the topsheet used in the diaper of FIG. 1.

FIG. 5(a) and FIG. 5(b) are each a schematic cross-sectional view taken along line Vy-Vy and line Vx-Vx, respectively, of FIG. 4.

FIG. 6 is a fragmentary schematic plan view of the skin facing side of another embodiment of the topsheet according to the invention (corresponding to FIG. 4).

FIG. 7 is a schematic cross-sectional view taken along line Vy-Vy of FIG. 6.

### DESCRIPTION OF EMBODIMENTS

The invention relates to an absorbent article of which the topsheet adapted to contact the skin of a wearer is soft and less likely to lift off during use.

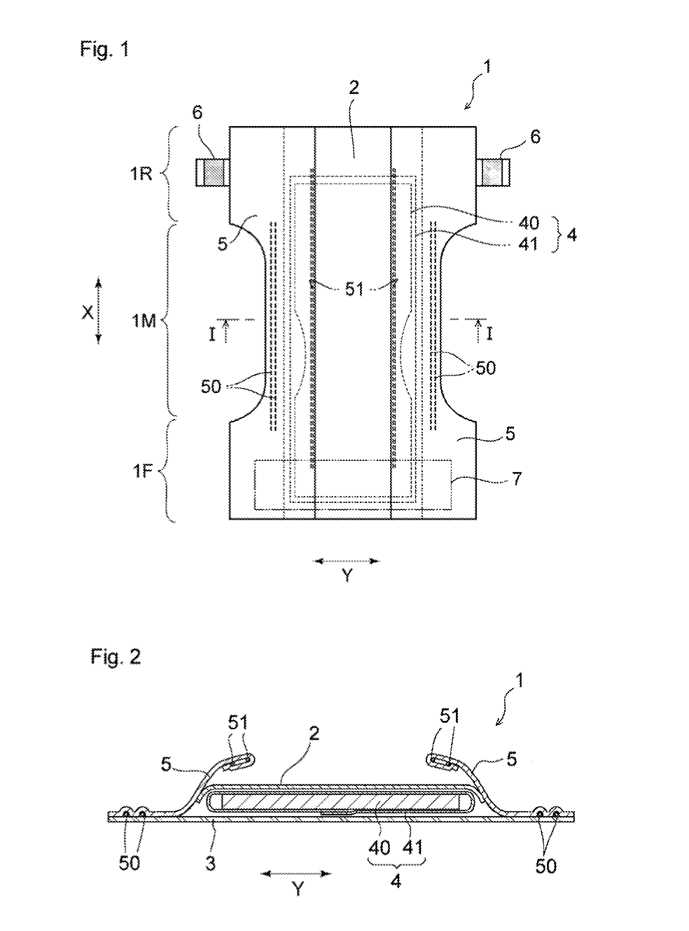

The absorbent article of the invention will be illustrated on the basis of its preferred embodiments with reference to the accompanying drawings. FIGS. 1 and 2 illustrate a disposable diaper 1 as an embodiment of the absorbent article according to the invention. The diaper 1 has a front portion 1F adapted to be worn about the front of a wearer, a rear portion 1R adapted to be worn about the back of a wearer, and a crotch portion 1M intermediate the front portion 1F and the rear portion 1R. The diaper 1 has a longitudinal direction X corresponding to the front-to-back direction of a wearer and extending from the front portion 1F to rear portion 1R through the crotch portion 1M and a lateral direction Y perpendicular to the longitudinal direction X. The crotch portion 1M has a target zone that is to face a wearer's point of body fluid discharge, such as the penis of a wearer, when worn. The target zone is usually located in or near the longitudinal middle portion of the diaper 1.

As illustrated in FIGS. 1 and 2, the diaper 1 includes an absorbent member 4, a liquid permeable topsheet 2 disposed on the skin facing side of the absorbent member 4 and adapted to contact the skin of a wearer, and a liquid impermeable or water repellent backsheet 3 disposed on the non-skin facing side of the absorbent member 4. In plan view, as illustrated in FIG. 1, the diaper 1 has the shape of an oblong hourglass that is longer in the longitudinal direction X than in the lateral direction Y and has a narrowed longitudinal middle portion located in the crotch portion 1M. Both the topsheet 2 and the backsheet 3 are larger in plan view than the absorbent member 4 interposed therebetween. The backsheet 3 is larger in plan view than the topsheet 2 and extends outward from the perimeter of the absorbent member 4 to define the outline of the diaper 1 in its flat-out and uncontracted state as illustrated in FIG. 1. The backsheet 3 may be of any material conventionally used in this type of absorbent articles, such as resin film or nonwoven fabric laminated with resin film.

As used herein, the term “skin facing side” refers to the side of an absorbent article or a member (for example, a topsheet 2) constituting the absorbent article that faces the wearer's skin while worn, i.e., one of the two sides that is closer to the skin than the other. The term “non-skin facing side” refers to the side of an absorbent article or a member constituting the absorbent article that faces away from the wearer's skin (or faces a garment) while worn, i.e., one of the two sides that is farther from the skin than the other. As used herein, the expression “while worn” means the state of an absorbent article being applied to the body in a normal appropriate position, in other words, in the right position and does not include a state of an absorbent article shifted from the right position.

The absorbent member 4 is oblong in the longitudinal direction X in plan view as illustrated in FIG. 1. The absorbent member 4 extends from the front portion 1F to the rear portion 1R. The absorbent member 4 includes a liquid retentive absorbent core 40 containing an absorbent material and a core wrap sheet 41 covering the skin facing side and the non-skin facing side of the absorbent core 40. The absorbent core 40 and the core wrap sheet 41 are bonded by a known bonding means, such as a hot-melt adhesive.

The absorbent core 40 has a single layer structure and is hourglass-shaped with its longitudinal middle portion narrowed in a plan view as shown in FIG. 1. The absorbent core 40 is an airlaid aggregate of core-forming material including an absorbent material. Any absorbent materials commonly used for making an absorbent core of this type may be used, including wood pulp, hydrophilic fibers such as synthetic fibers treated with a hydrophilizing agent, and absorbent particulate polymers. The absorbent core 40 may be an airlaid aggregate of hydrophilic fiber or an airlaid aggregate of hydrophilic fiber having an absorbent particulate polymer supported thereon.

The core wrap sheet 41 may be of a water permeable sheet material, such as paper or nonwovens. In the diaper 1 of the present embodiment, the core wrap sheet 41 is a single sheet of which the dimension in the lateral direction Y is two to three times the width (dimension in the lateral direction Y) of the absorbent core 40 enough to cover the entire area of the skin facing side of the absorbent core 40, extend laterally outward from the laterally opposing edges of the core 40, and wrap the underside of the core thereby covering the entire area of the non-skin facing side of the absorbent core 40 as illustrated in FIG. 2. The form of the core wrap sheet 41 is not limited to that of FIG. 2 and may be a combination of a first core wrap sheet covering the skin facing side of the absorbent core 40 and a second core wrap sheet covering the non-skin facing side of the core 40, the first core wrap sheet and the second core wrap sheet being separate.

The diaper 1 further includes a side sheet 5 disposed on either lateral side portion thereof on the side of the topsheet 2 to extend in the longitudinal direction X. Each side sheet 5 has a proximal longitudinal edge and a distal longitudinal edge locating laterally outward than the proximal longitudinal edge. In plan view as in FIG. 1, the proximal edge overlaps the absorbent member 4, and the distal edge extends laterally outward from the side edge of the absorbent member 4 and is bonded to the backsheet 3 as illustrated in FIG. 2. Thread-like elastic members 50 are fixed in their stretched state between each side sheet 5 and the backsheet 3 to extend in the longitudinal direction X in the leg portion adapted to be worn around the leg of a wearer. The leg portions form a pair of leg cuffs due to contraction of the elastic members 50 while in use. Thread-like elastic members 51 are fixed in their stretched state along the longitudinally extending proximal edge of each side sheet 5. The elastic members 51 contract while in use to make the side sheet 5 rise from the bond with the backsheet 3 toward the wearer's skin in at least the crotch portion 1M to form a leak-proof cuff. The leak-proof cuff is able to block the laterally outward leakage of a discharged fluid, such as urine. The topsheet 2, backsheet 3, absorbent member 4, pair of side sheets 5, and elastic members 50 and 51 are bonded to their respective adjoining members by a known bonding means, such as a hot-melt adhesive.

The diaper 1 is an open-style disposable diaper and is provided on each side edge of its rear portion 1R with a fastening tape 6 as illustrated in FIG. 1. Each fastening tape 6 has an unshown attachment portion formed of a male component of a mechanical fastener. The front portion 1F of the diaper 1 is provided on its non-skin facing side with a landing zone 7 formed of a female component of the mechanical hook and loop fastener. The landing zone 7 is formed by bonding and fixing the female component of the mechanical hook and loop fastener to the non-skin facing side of the backsheet 3 of the front portion 1F by a known bonding means, such as adhesive bonding or heat sealing, so that the attachment portion of the fastening tape 6 may be releasably secured thereto.

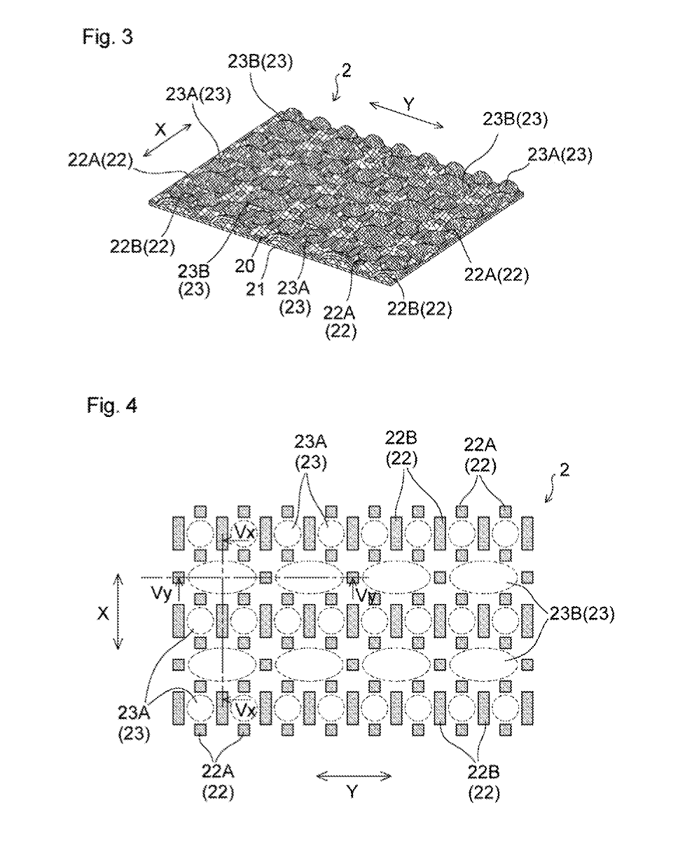

The topsheet 2 will be described in more detail. FIGS. 3 to 5 are enlarged fragmentary views of the topsheet 2. The topsheet 2 includes a stack of a first sheet 20 and a second sheet 21. The first sheet 20 and the second sheet 21 are bonded to each other at a plurality of bonds 22 formed by embossing. The first sheet 20 protrudes away from the second sheet 21 at portions other than the bonds 22 to form a plurality of projections 23 toward the skin of a wearer.

The topsheet 2 has the first sheet 20 on its side closer to the wearer's skin and the second sheet 21 on its side farther from the wearer's skin. The sheets 20 and 21 are joined together in parts in their thickness direction by a large number of bonds 22. In the first sheet 20, the portion between every pair of adjacent bonds 22 is protuberant upward to form a projection 23 on the skin facing side of the topsheet 2. Thus, the topsheet 2 has an undulating textured surface with projections and depressions on the skin facing side while having a substantially flat surface on the non-skin facing side, i.e., the side facing the absorbent member 4. Bonding of the sheets 20 and 21 to form the bonds 22 may be achieved by any known embossing processing, such as embossing with or without heat or ultrasonic embossing.

The topsheet 2 has, in its plane, a first direction X and a second direction Y perpendicular to the first direction X. With the topsheet 2 assembled into the diaper 1, the first direction X is coincident with the longitudinal direction X of the diaper 1, and the second direction Y is coincident with the lateral direction Y of the diaper 1.

In general, the first direction X of the topsheet 2 is often coincident with the machine direction in which the first sheet 20 and the second sheet 21 are each fed from their respective rolls and transported to make the topsheet 2. In using nonwovens as the first sheet 20 and the second sheet 21, generally, the first direction X of the topsheet 2 is often coincident with the dominant orientation direction of the fibers making up the nonwovens.

In the topsheet 2 in the embodiment, the bonds 22 include a plurality of first bonds 22A having a square plan-view shape and a plurality of second bonds 22B having a rectangular plan-view shape. The second bond 22B is larger in area than the first bond 22A and the longitudinal direction thereof is coincident with the first direction X. Since the first sheet 20 and the second sheet 21 are pressed unitedly at bonds 22 (including the bonds 22A and 22B) by embossing, the first sheet 20 and the second sheet 21 are densified to have high densities at every bond 22 than in non-bonded portions. Preferably, the first sheet 20 and the second sheet 21 are fusion bonded to each other as a result of the fiber-forming resin of at least one of the two sheets being melted and solidified.

In the embodiment, the first bonds 22A and the second bonds 22B are formed in a staggered pattern. That is, as illustrated in FIG. 4, the plurality of first bonds 22A are spaced in the lateral direction Y in straight rows, and the plurality of second bonds 22B are also spaced in the lateral direction Y in straight rows. The rows of the first bonds 22A and the rows of the second bonds 22B alternate in the longitudinal direction X such that the bonds 22A and 22B in adjacent rows do not align in the longitudinal direction X.

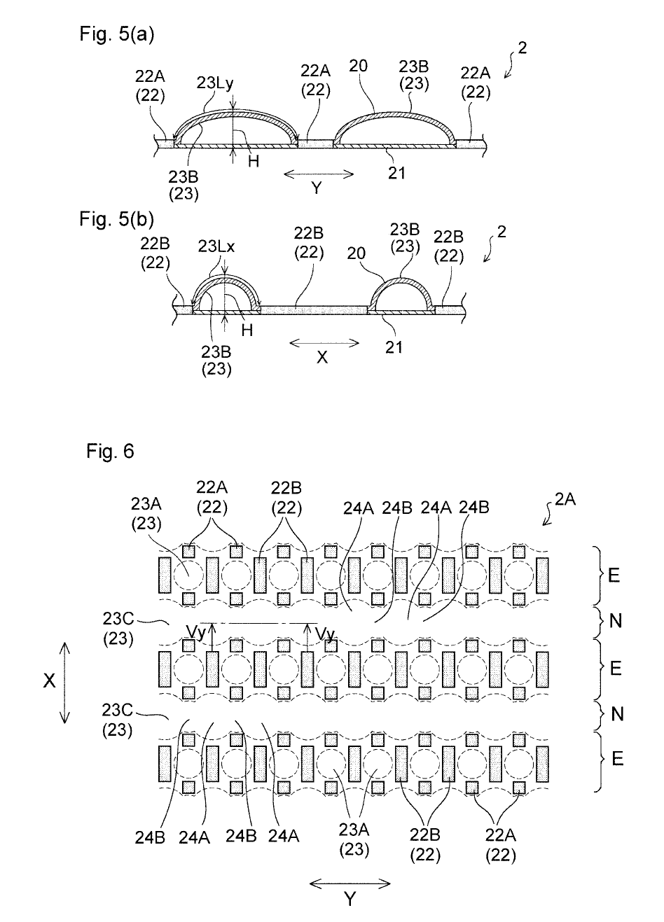

In the embodiment, the projections 23 of the topsheet 2 include first projections 23A that are circular in plan view and second projections 23B that have an elliptic plan-view shape of which the longer axis is coincident with the second direction Y. The second projection 23B is larger in area than the first projection 23A. Each of the first projections 23A and the second projections 23B is formed in a region surrounded by a plurality of bonds 22 (debosses or depressions) and may therefore be called a projection in a bond-surrounded region. The first projections 23A and the second projections 23B each have a top in a cross-section taken in each of the first direction X and the second direction Y. In the embodiment, the second projections 23B are hollow as illustrated in FIG. 5, and the first projections 23A, while not shown, are also hollow. However, the structure of the projections 23 is not limited to that of the embodiment. For example, the projections 23A and/or 23B do not need to be hollow and may have a solid structure filled with a material forming the topsheet 2.

The second projections 23B have a larger height H (see FIG. 5) than the first projections 23A and are the highest of all the projections including the first projections 23A and the second projections 23B of the topsheet 2. The term “height” as used herein with respect to the projections denotes the distance from the non-skin facing surface of the second sheet 21 to the skin-facing surface of the first sheet 20 at a position where the first sheet 20 protrudes farthest away from the second sheet 21 in the projection 23 (23A or 23B), namely the apparent thickness of that position. While FIG. 5 shows only the second projections 23B, the same definition of “height” applies to the first projections 23A. The ratio of the height of the second projections 23B to that of the first projections 23A, second projections 23B/first projections 23A, is preferably 1.1 or higher, more preferably 1.3 or higher, preferably 5.0 or lower, more preferably 2.0 or lower.

In the embodiment, the plurality of first projections 23A are spaced in the lateral direction Y in straight rows. The plurality of second projections 23B are also spaced in the lateral direction Y in straight rows. The rows of first projections 23A and the rows of second projections 23B alternate in the longitudinal direction X in such a pattern that one pair of adjacent first projections 23A and one second projection 23B are aligned in the longitudinal direction X.

In the embodiment, the plurality of bonds 22, including 22A and 22B, are arranged in a discrete pattern in plan view as illustrated in FIG. 4. That is, the plurality of bonds 22 are arranged discretely in a regular pattern over the whole area of the topsheet 2, whereby the plurality of projections 23, including 23A and 23B, are each surrounded by at least two bonds 22. The bonds 22 that surround a single projection 23 are spaced from each other around the projection 23. The individual projections 23 are preferably surrounded by more than two bonds 22 with a view to make the protrusion more distinct, and in this case, it is preferred for the individual projections 23 to have its top positioned at substantially the center of the region surrounded by the more than two bonds 22. In the embodiment, the first projections 23A with a circular plan-view shape are each surrounded by a total of four bonds 22, two first bonds 22A aligned in the longitudinal direction X and two second bonds 22B aligned in the lateral direction Y. The second projections 23B with an elliptic plan-view shape are each surrounded by six first bonds 22A.

At the bonds 22 (22A and 22B), the sheet-forming material of the topsheet 2 is densified by embossing, and therefore the bonds 22 have higher densities and are stiffer than the surrounding regions. Since such relatively hard bonds 22 are formed discretely in plan view as illustrated in FIG. 4, the individual projections 23 (23A and 23B) are not encircled by a continuous hard portion. Therefore, the projections 23 exhibits further improved softness and have increased freedom of shape deformation. In contrast to this, when a plurality of bonds 22 each have a linear plan-view shape and intersect one another, forming a grid pattern, the projection 23 is formed in each cell of the grid. In such a case, a continuous hard region formed of the linear bonds 22 continuously encircles all round each projection 23. The continuous arrangement of the bonds 22 is therefore disadvantageous compared with the discrete arrangement in terms of softness and freedom of shape deformation of the projections 23.

One of the main characteristics of the topsheet 2 according to the embodiment consists in that all the conditions (1) to (4) described below are satisfied. The conditions (1) to (4) are essential for the topsheet to be soft, less likely to lift off during use, and capable of greatly improving comfort experienced by a wearer when using an absorbent article such as the diaper 1. More specifically, to satisfy the conditions (1) and (2) means a combination of relatively large projections and a relatively small number of bonds (debosses), which provides good deformability and softness of the projections, and to satisfy the conditions (3) and (4), which specify the area ratio of the bonds and the shear strength of the topsheet, promises softness and prevention of the topsheet lifting off.

(1) The projections 23 have a height H (see FIG. 5) of 0.5 to 10 mm.
(2) The bonds 22 have a density (the sum of the total number of the first bonds 22A and the total number of the second bonds 22B) of 10 to 30 bonds per a randomly chosen rectangular region of the topsheet 2 measuring 10 mm in length and 15 mm in width in plan view.

(3) The bonds 22 in the rectangular region defined above have a total area ratio (the ratio of the sum of the total area of the first bonds 22A and the total area of the second bonds 22B; hereinafter also referred to as “bond area ratio”) of 12% to 35% relative to the area of the rectangular region.

(4) The topsheet has a shear strength of 21 to 70 N.

The condition (1) is made with the aim mainly of improving deformability and softness of the topsheet 2. With the height H of the projections 23 of 0.5 mm or greater, the aim is accomplished. With the height H of 10 mm or smaller, the projections 23 are less likely to cling to the skin, thereby reducing the cause of wearer discomfort. The height H of the projections 23 is preferably 0.6 mm or greater and 5 mm or smaller. The heights H of the projections 23 are measured as follows.

### Method for Measurement of Height of Projections:

The height of the projections of the topsheet under examination is measured by observing a cross-section of the topsheet under a microscope. Specifically, a cross-section is magnified to a degree that provides a field of view in which projections and their surroundings are sufficiently observable and measurable (e.g., 10 to 100 times), and the heights of the projections in the field, i.e., the height corresponding to the height H of the projections 23B (the second projections 23B) illustrated in FIG. 5 (the distance from the non-skin facing surface of the second sheet 21 to the skin-facing surface of the first sheet 20 measured at a position where the first sheet 20 protrudes farthest away from the second sheet 21 in the projection 23, namely the apparent thickness of that position) is measured using a digital microscope VHX-1000 from Keyence.

The condition (2) is made with the view mainly of increasing the freedom of shape deformation of the projections 23 (23A and 23B) thereby allowing the projections 23 to change their contour on contact with the wearer's skin during use of the topsheet 2. The condition (2) combined with the condition (1) allows for forming relatively large projections 23 with a relatively small number of bonds 22. If the total number (density) of the bonds 22 formed in the above-defined region in the topsheet 2 is smaller than 10, the topsheet 2 (the first sheet 20) tends to lift off in use. If the total number is greater than 30, the topsheet 2 tends to have reduced softness. The total number of the bonds in the above-defined region is preferably 15 or more, more preferably 20 or more, and preferably 28 or less, more preferably 25 or less.

The condition (3) is made with the aim mainly of preventing the peel (shedding of fuzz) of the first sheet 20 at the bonds 22 (22A and 22B) and the resulting lift-off of the topsheet 2. The aim is accomplished with the bond area ratio of 12% or higher. As long as the bond area ratio is 35% or lower, the topsheet 2 exhibits high softness. The bond area ratio is preferably 15% or higher, more preferably 20% or higher, preferably 30% or lower, more preferably 25% or lower.

The condition (4) is made with the same aim of making the condition (3). The aim is achieved when the topsheet 2 has a shear strength of 21 N or higher. With a shear strength of 70 N or lower, the topsheet 2 has high softness. The shear strength is preferably 23 N or higher, more preferably 24 N or higher, even more preferably 26.5 N or higher, preferably 40 N or lower, more preferably 30 N or lower, even more preferably 28 N or lower. The shear strength of the topsheet 2 is generally influenced by factors such as the arrangement pattern of the bonds 22, the bond strength of the bonds 22, and the fiber material making up the topsheet 2. Therefore, the shear strength of the topsheet 2 can be adjusted within the above specified range by appropriate selection of these factors. The shear strength is determined as follows.

### Method for Determining Shear Strength of Topsheet:

A rectangular specimen of 30 mm in width and 50 mm in length in plan view is cut out of a topsheet under examination. A double-sided adhesive tape (product name: No. 500, from Nitto Denko Corp.) of the same shape and size as the specimen is stuck on a PET film (product name: Star OHP Film, from Star Corp.) to obtain laminate (A). The specimen is stuck on its non-textured side to the other side of the adhesive tape of laminate (A) in superposed relation to obtain laminate (B), and laminate (B) is rolled back and forth five times with a 1 Kg roller to ensure firm adhesion of the specimen and the adhesive tape in laminate (B). As another adherend, a male component of a mechanical fastener (product name: 1600PPI, from 3M) of a shape and size enough to cover the entire area of the specimen is attached on its hook side to the textured side of the specimen in laminate (B) and pressed with a load of 1.5 kPa. The resulting specimen assembly is set on a tensile tester (Augograph AG-X, from Shimadz Corp.) at an initial jaw spacing of 150 mm, and 180 degree peel test was performed on the specimen assembly. In the test, one of the longitudinal ends of laminate (B) is clamped into one of the jaws of the tester and the other end of the adherend into the other jaw, and the specimen assembly is pulled at 180 degree angle at a rate of 300 mm/min. The maximum tensile load exerted is recorded. A specimen is taken from the laterally left, middle, and right portions in each of the front and rear portions of a diaper to make a total of six specimens. Such six specimens per diaper are taken from three diapers. The average of the measured values shall define the shear strength (unit: N) of the topsheet.

The topsheet 2 of the diaper 1 of the embodiment has the following characteristic in addition to the conditions (1) to (4). The second projections 23B, which are the highest of all projections (23A and 23B) of the topsheet 2 as stated earlier, have a length 23Ly in the lateral direction Y (the second direction) and a length 23Lx in the longitudinal direction X (the first direction), both measured along the outer surface of the individual second projections 23B, and the length 23Ly is longer than the length 23Lx, as illustrated in FIG. 5. This geometric characteristic offers the advantage that the second projections 23B (the highest projections) are easily deformable to make the topsheet 2 feel softer, on the basis of the fact that, while the diaper 1 is worn, the friction between the skin and the diaper 1 occurs principally in the longitudinal direction X.

In order to further ensure the above described effect and advantage, the ratio of the length 23Ly (in the lateral direction Y) to the length 23Lx (in the longitudinal direction X), 23Ly/23Lx, as measured along the outer surface of the projection 23B is preferably 1.1 or higher, more preferably 1.2 or higher, preferably 5.0 or lower, more preferably 3.0 or lower.

The length 23Lx of the second (highest) projection 23B measured in the longitudinal direction X along the outer surface of the projection is preferably 1.5 rum or greater, more preferably 2.0 mm or greater, preferably 10 mm or smaller, more preferably 6 mm or smaller.

The length 23Ly of the second (highest) projection 23B measured in the lateral direction Y along the outer surface of the projection is preferably 2.0 mm or greater, more preferably 2.5 mm or greater, preferably 10 mm or smaller, more preferably 6 mm or smaller.

The lengths of the projections 23 along the outer surface are measured as follows.

### Method of Measurement of Length Along Outer Surface of Projection:

The lengths along the outer surface of the projections of the topsheet under examination are measured by observing a cross-section of the topsheet under a microscope. Specifically, a cross-section of the topsheet is magnified to a degree that provides a field of view in which projections and their surroundings are sufficiently observable and measurable (e.g., 10 to 100 times), and the length of the projection in a predetermined direction in the field, i.e., the length corresponding to the length 23Lx of the projection 23 (the second projection 23B) in the longitudinal direction X or the length corresponding to the length 23Ly of the projection 23 in the lateral direction Y (see FIG. 5), is measured along the outer surface of the projections, using a digital microscope VHX-1000 from Keyence. As used herein, the phrase “length (measured) in the longitudinal direction X along the outer surface of the projection” refers to the length of the outer surface of the part of a projection 23 that is protuberant away from the part of the second sheet 21 that is located between two bonds 22 having the projection 23 therebetween in the longitudinal direction X as illustrated in FIG. 5(b). Likewise, the phrase “length (measured) in the lateral direction Y along the outer surface of the projection” refers to the length of the outer surface of the part of a projection 23 that is protuberant away from the part of the second sheet 21 that is located between two bonds 22 having the projection 23 therebetween in the lateral direction Y as illustrated in FIG. 5(a).

The materials of the topsheet 2 will then be described. The first sheet 20 and the second sheet 21 for the topsheet 2 are each formed of a sheet material. The sheet material may be a fiber sheet, such as nonwoven, woven, and knitted fabric, or resin film. Fiber sheets are preferred in view of feel to the touch. Nonwoven fabric is particularly preferred. The first sheet 20 and the second sheet 21 may be made of the same material or different materials. They are preferably of the same material so as to ensure the adhesion strength of the bonds 22 formed of the two sheets 20 and 21.

Examples of the nonwoven fabric that can be used to form the first and second sheet 20 and 21 include air-through nonwovens, spun-bonded nonwovens, hydroentangled nonwovens, melt-blown nonwovens, resin-bonded nonwovens, and needle-punched nonwovens. The sheet material may be a composite sheet of two or more of these nonwoven fabrics or a composite sheet of the nonwoven fabric and resin film. Preferred of these sheet materials are air-through nonwovens and spun-bonded nonwovens. The first sheet 20, in particular, which is to face the wearer's skin while the diaper 1 is worn, is preferably formed of air-through nonwoven fabric. The basis weight of the nonwoven fabric is preferably 10 g/m² or more, more preferably 10 g/m² or more, preferably 100 g/m² or less, more preferably 30 g/m² or less.

The fiber making up the nonwoven fabric for the sheets 20 and 21 may be of various kinds of thermoplastic resins (fusible fibers), which are exemplified by polyolefins, such as polyethylene and polypropylene, polyesters, such as polyethylene terephthalate, polyamides, such as nylon 6 and nylon 66, polyacrylic acid, poly(alkyl methacrylates), polyvinyl chloride, and polyvinylidene chloride. These resins may be used either individually or in combination of two or more thereof. The fusible fibers may be staple fibers or filament fibers and may be hydrophilic or water repellant. The fusible fibers may be in the form of conjugated fibers having, for example, a sheath/core configuration or a side-by-side configuration, splittable fibers, modified cross-section fibers, or heat shrinkable fibers. These fibers may be used either individually or in combination of two or more thereof. The two sheets 20 and 21 may be the same or different in composition. The phrase “same in composition” is intended to mean that the two sheets are made of the same material and have the same content of the material whether or not they are different in appearance, such as dimensions or plan-view shape.

Preferred examples of the nonwoven fabric forming the sheets 20 and 21 include nonwoven fabric containing one or more kinds of fusible conjugate fibers composed of two or more resins, such as nonwoven fabric containing two kinds of fusible sheath/core conjugate fibers. It is preferable that the two kinds of sheath/core conjugate fibers be different in kind of sheath component, or have the same sheath component and the same core component but different in volume ratio of the core component and the sheath component.

It is preferred for the topsheet 2 to have a large number of intersections of the fusible conjugate fibers (preferably fusible sheath/core conjugate fibers) and for the fusible conjugate fibers to be fusion-bonded to each other at at least part of their intersections. The presence of a large number of such fused intersections between the fusible conjugate fibers in the topsheet 2 makes it possible to control the characteristics of the topsheet 2, such as softness, through the adjustment of the number of the fused intersections or the fusion bond strength between the fibers of the fused intersections. The characteristics of the topsheet 2, such as softness, can thus be improved further.

With the view of forming a large number of fused intersections between the fusible conjugate fibers, it is preferred that the topsheet 2 contain at least two kinds of fusible conjugate fibers. In particular, when each of the first sheet 20 and the second sheet 21 for the topsheet 2 is air-through nonwoven fabric prepared by blowing hot air through a carded web formed of two or more kinds of fusible conjugate fibers to cause the fibers to be fusion bonded together at their intersections, it is easier to control the number of the fused intersections of the constituent fibers (fusible conjugate fibers) or the fusion bond strength of the fibers at the fused intersections because the fusion bondability between the constituent fibers varies with the combination of the fibers. This will lead to still further improvement on the softness of the topsheet 2. The fusible conjugate fibers (preferably fusible sheath/core conjugate fibers) may be present in one or both of the sheets 20 and 21 forming the topsheet 2. It is preferred for the fusible conjugate fibers to be present in at least the first sheet 20, which is located closer to the wearer's skin and required to be soft.

The sheets 20 and 21 may contain crimped fusible fibers (self-crimping fusible fibers having developed crimps). In one example, the self-crimping fusible fibers may be eccentric sheath/core or side-by-side bicomponent conjugate fibers composed of two thermoplastic polymers having different shrinkage percentages. A preferred example of the two thermoplastic polymers having different shrinkage percentages is a combination of an ethylene-propylene random copolymer (EP) and polypropylene (PP).

The topsheet 2 having the aforementioned configuration can be produced by, for example, the method for making a composite sheet disclosed in JP 2015-112343A. Specifically, the first sheet 20 of continuous length is introduced into the bite of intermeshing first and second rollers to be three-dimensionally textured. The textured first sheet 20 is released from the bite between the rollers while being held onto the periphery of the first roller, and the second sheet 21 is fed to join the first sheet 20. The two sheets 20 and 21 are pressed to each other under heat between the projections on the first roller and a heat roller to bond them to each other in parts. The shape of the intermeshing projections and depressions on the first and second rollers and the pattern of the bonds formed between the first roller and the heat roller are varied between the middle region and pair of side regions of the first sheet 20. When the first sheet 20 is fed into the bite between the first and second rollers to be three-dimensionally textured, it is preferred that the first sheet 20 be sucked toward the inside of the roller to help three-dimensional texturing of the first sheet 20.

FIG. 6 shows a topsheet 2A according to another embodiment of the topsheet according to the invention. The description of the topsheet 2A in FIG. 6 will generally be confined to the difference from the topsheet 2 (see FIGS. 3 through 5). Other similar parts are indicated by similar numerals and will not be redundantly described. Unless the context is specifically otherwise, the description of the topsheet 2 applies equally to the topsheet 2A.

As illustrated in FIG. 6, the topsheet 2A has bond-free, continuously raised portions N extending in the second direction Y, i.e., the lateral direction Y of the diaper 1, and discontinuously raised portions E, which are discontinued by regularly spaced bonds 22, extending in the lateral direction Y. The continuously raised portions N and the discontinuously raised portions E alternate in the first direction X, i.e., the longitudinal direction X of the diaper 1.

The discontinuously raised portion E has bonds (debosses) 22 formed by bonding the first and second sheets 20 and 21 in parts by embossing. The bonds 22 include a plurality of first bonds 22A having a square plan-view shape and a plurality of second bonds 22B having a rectangular plan-view shape. The discontinuously raised portion E has, as the projection 23, a hollow projection 23A having a circular plan-view shape in every region surrounded by these bonds 22A and 22B, i.e., a bond-surrounded region. The projections 23A in a bond-surrounded region are discontinuously formed, being spaced at a predetermined interval in the lateral direction Y in straight rows. The configuration of the discontinuously raised portion E in the topsheet 2A is substantially the same as that of the laterally extending row of the first projections 23A and the neighboring bonds 22A and 22B in the topsheet 2 (see FIG. 4).

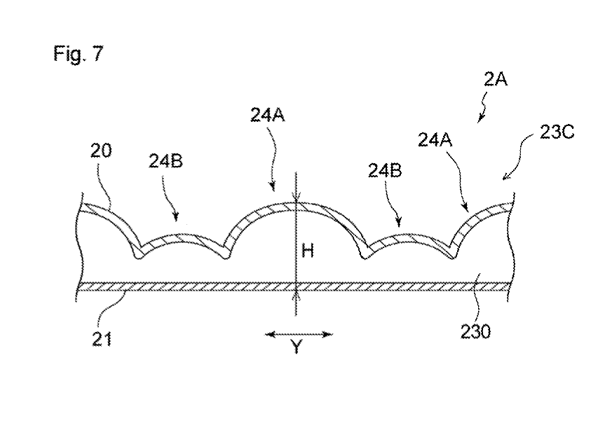

On the other hand, the continuously raised portion N has, as a projection 23, a continuous projection 23C continuously extending in the lateral direction Y. As illustrated in FIGS. 6 and 7, the continuous projection 23C is composed of sub-projections 24A and 24B connected to each other. As used herein, the expression “connected to each other” with respect to projections means that two adjacent projections (sub-projections) are not separated from each other by the bond 22, and that part of one of them forms part of the other and vice versa.

In the embodiment, as illustrated in FIG. 7, two kinds of sub-projections different in height, i.e., sub-projections 24A and sub-projections 24B alternate in the lateral direction Y and connect to each other to form the continuous projection 23C. The sub-projections 24A are relatively high, while the sub-projections 24B are relatively low. In accordance with the above described definition of the term “height” with respect to the projections, the height of the continuous projection 23C is the height of the sub-projections 24A, i.e., the separation distance between the two sheets 20 and 21 measured at a position where the first sheet 20 protrudes farthest away from the second sheet 21 in the sub-projections 24A. The sub-projections 24A and 24B are almost circular in plan view and have their respective tops in a cross-section taken in each of the first direction X and the second direction Y. The continuous projection 23C is hollow, and the hollow 230 thereof continuously extends substantially the whole length of the continuous projection 23C in the lateral direction Y. The continuous projection 23C may continuously extend the whole width (dimension in the lateral direction Y) of the topsheet 2A, or the continuously raised portion N may have two or more continuous projections 23C arranged at a predetermined interval in the lateral direction Y in a row. In the latter case, a bond 22 is formed between laterally adjacent continuous projections 23C.

While the invention has been described on the basis of its embodiments, the invention is not construed as being limited thereto, and various changes and modifications can be made therein without departing from the spirit and scope of the invention. For example, a liquid permeable, rewet barrier layer, which is also called a sublayer, may be interposed between the topsheet 2 and the absorbent member 4 (specifically, the core wrap sheet 41) for the purpose of preventing retransmission of discharged body fluids from the absorbent core 40 to the wearer's skin, namely rewet. While the bonds 22 shown in FIGS. 4 and 6 have a quadrilateral plan-view shape, the plan-view shape of the bonds 22 is not limited thereto and may be a circular, polygonal (such as triangular, or hexagonal), elliptic, or cross shape, or a combination of these shapes. The pattern of arrangement of the bonds 22 is not limited to the staggered pattern as illustrated in FIG. 4 and may be altered as appropriate.

The absorbent article of the invention is not limited to open-type disposable diapers of the foregoing embodiments and includes in its scope a broad range of articles used to absorb body fluids discharged from human bodies, such as urine, menstrual blood, soft stools, and sweat, including pull-on type disposable diapers, sanitary napkins, sanitary panties, and so on. The following clauses are also disclosed with respect to the above described embodiments of the absorbent article of the invention.

1. An absorbent article comprising an absorbent member and a topsheet on the skin facing side of the absorbent member and having a longitudinal direction corresponding to the front-to-back direction of a wearer and a lateral direction perpendicular to the longitudinal direction,

the topsheet comprising a stack of a first sheet and a second sheet bonded to each other at a plurality of bonds,

the first sheet having a plurality of projections toward the skin of a wearer resulting from the first sheet protruding away from the second sheet at portions other than the bonds, and, the topsheet satisfying the following conditions (1) to (4):

(1) the projections having a height of 0.5 to 10 mm,

(2) the bonds having a density of 10 to 30 bonds per a randomly chosen rectangular region of the topsheet measuring 10 mm in length and 15 mm in width in plan view,

(3) the bonds in the rectangular region having a total area ratio of 12% to 35% relative to the area of the rectangular region, and

(4) the topsheet having a shear strength of 21 to 70 N.

2. The absorbent article as set forth in clause 1, wherein the plurality of bonds are arranged in a discrete pattern in plan view.
3. The absorbent article as set forth in clause 2, wherein the plurality of bonds are arranged discretely in a regular pattern over the whole area of the topsheet such that the plurality of projections are each surrounded by at least two of the plurality of bonds spaced from each other around each projection.
4. The absorbent article as set forth in clause 2 or 3, wherein the projections are each surrounded by more than two of the plurality of bonds and have their respective tops positioned at substantially the center of the region surrounded by the more than two bonds.
5. The absorbent article as set forth in any one of clauses 1 to 4, wherein the projections have a height of 0.6 to 5 mm.
6. The absorbent article as set forth in any one of clauses 1 to 5, wherein the density of the bonds per rectangular region defined in claim 1 is preferably 15 or more, more preferably 20 or more, and preferably 28 or less, more preferably 25 or less.
7. The absorbent article as set forth in any one of clauses 1 to 6, wherein the bonds in the rectangular region defined in claim 1 has a total area ratio of preferably 15% or higher, more preferably 20% or higher relative to the area of the rectangular region, and of preferably 30% or lower, more preferably 25% or lower relative to the area of the rectangular region.
8. The absorbent article as set forth in any one of clauses 1 to 7, wherein the shear strength is preferably 23 N or higher, more preferably 24 N or higher, even more preferably 26.5 N or higher, and preferably 40 N or lower, more preferably 30 N or lower, even more preferably 28 N or lower.
9. The absorbent article as set forth in any one of clauses 1 to 8, wherein the topsheet comprises fusible conjugate fibers made up of more than one resin and has a large number of intersections of the fusible conjugate fibers, the fusible conjugate fibers being fusion-bonded to each other at at least part of their intersections.
10. The absorbent article as set forth in any one of clauses 1 to 9, wherein the plurality of bonds comprise a plurality of first bonds having a square plan-view shape and a plurality of second bonds having a rectangular plan-view shape.
11. The absorbent article as set forth in clause 10, wherein each second bond is larger in area than the first bond and longer in the longitudinal direction than in the lateral direction.
12. The absorbent article as set forth in clause 10 or 11, wherein the plurality of first bonds are spaced in the lateral direction in straight rows, and the plurality of second bonds are spaced in the lateral direction in straight rows, the rows of the first bonds and the rows of the second bonds alternating in the longitudinal direction such that the first bonds and second bonds in adjacent rows do not align in the longitudinal direction.
13. The absorbent article as set forth in any one of clauses 10 to 12, wherein the plurality of projections comprise a first projection having a circular plan-view shape and a second projection having an elliptic plan-view shape of which the longer axis is coincident with the lateral direction, and the second projection is larger in area than the first projection.
14. The absorbent article as set forth in clause 13, wherein the second projection is higher than the first projection and the highest of all the plurality of projections.
15. The absorbent article as set forth in clause 13 or 14, wherein a ratio of the height of the second projection to that of the first projection, second projection/first projection, is preferably 1.1 or higher, more preferably 1.3 or higher, preferably 5.0 or lower, more preferably 2.0 or lower.
16. The absorbent article as set forth in any one of clauses 13 to 15, wherein the first projection comprises a plurality of first protrusions, and the second projection comprises a plurality of second protrusions, the plurality of first projections being spaced in the lateral direction in straight rows, the plurality of second projections being spaced in the lateral direction in straight rows, and the rows of first projections and the rows of second projections alternating in the longitudinal direction in such a pattern that one pair of adjacent first projections and one second projection are aligned in the longitudinal direction.
17. The absorbent article as set forth in any one of clauses 13 to 16, wherein the first projection is surrounded by a total of four bonds consisting of two first bonds aligned in the longitudinal direction and two second bonds aligned in the lateral direction, and the second projection is surrounded by six first bonds.
18. The absorbent article as set forth in any one of clauses 1 to 17, wherein the highest projection of the plurality of projections has a length in the longitudinal direction and a length in the lateral direction that is longer than the length in the longitudinal direction, both measured along the outer surface of the projection.
19. The absorbent article as set forth in clause 18, wherein the highest projection has a ratio of the length in the lateral direction to the length in the longitudinal direction, lateral direction/longitudinal direction, of preferably 1.1 or higher, more preferably 1.2 or higher, and of preferably 5.0 or lower, more preferably 3.0 or lower.
20. The absorbent article as set forth in clause 18 or 19, wherein the highest projection has a length of preferably 1.5 mm or greater, more preferably 2.0 mm or greater, and preferably 10 mm or smaller, more preferably 6 mm or smaller measured along the outer surface thereof in the longitudinal direction.
21. The absorbent article as set forth in any one of clauses 18 to 20, wherein the highest projection has a length of preferably 2.0 mm or greater, more preferably 2.5 mm or greater, and preferably 10 mm or smaller, more preferably 6 mm or smaller measured along the outer surface thereof in the lateral direction.
22. The absorbent article as set forth in any one of clauses 1 to 21, wherein the absorbent member or an absorbent core as a main component of the absorbent core has a single layer structure and being hourglass-shaped with its longitudinal middle portion narrowed in plan view.

### EXAMPLES

The invention will now be illustrated in greater detail by way of Examples, but it should be understood that the invention is not limited thereto.

### Examples 1 to 3

An open-type disposable diaper having the same basic structure as that illustrated in FIGS. 1 and 2 was made. A sheet prepared by the method described below was used as a topsheet. A moisture permeable film having a basis weight of 20 g/m² was used as a backsheet. An airlaid pulp fiber pad having a basis weight of 210 g/m² and having 190 g/m² of an absorbent polymer (CAW101, from Nippon Shokubai Co., Ltd.) supported therein was used as an absorbent core. The absorbent core was covered on its entire surface with absorbent paper (core wrap sheet) having a basis weight of 14 g/m² to make an absorbent member measuring 360 mm in length (in the longitudinal direction) and 110 mm in width (in the lateral direction).

### Method for Making Topsheet Used in Examples:

Two sheets, a first sheet and a second sheet, were joined together by embossing in accordance with the method for making a composite sheet described in JP 2015-112343A cited supra, to thereby obtain a topsheet. The pattern of forming the bonds (projections) between the two sheets was varied in Examples 1 to 3 as described in Table 1 below. The first sheet, which was disposed closer to the wearer's skin than the other, was formed of air-through nonwoven fabric with a basis weight of 18 g/m². The second sheet, which was disposed farther from the wearer's skin than the other, was also formed of air-through nonwoven fabric with a basis weight of 18 g/m². The air-through nonwoven fabric for the first sheet had a dual layer structure composed of an upper layer (closer to the wearer's skin) and a lower layer (farther from the wearer's skin). The upper layer was made of polyethylene terephthalate core/polyethylene sheath conjugate fibers (resin mass ratio: 5/5, 2.3 dtex, core/sheath diameter ratio: 1.57). The lower layer was made of 70:30 (by mass) blended fiber of polyethylene core/polyethylene terephthalate sheath conjugate fibers (resin mass ratio: 5/5, 2.3 dtex, core/sheath diameter ratio: 1.57) and polyethylene core/polyethylene terephthalate sheath conjugate fibers (resin mass ratio: 2/8, 2.2 dtex, core/sheath diameter ratio: 1.42). The air-through nonwoven fabric for the second sheet was made of polyethylene terephthalate core/polyethylene sheath conjugate fibers (resin mass ratio: 5/5, 2.3 dtex).

The bond (projection) formation pattern in the topsheets used in Examples 1 and 2 was similar to the pattern in the topsheet 2A (see FIG. 6). Specifically, in the topsheets of Examples 1 and 2, continuously and discontinuously raised portions extending in the lateral direction alternated in the longitudinal direction X. Each discontinuously raised portion had a plurality of regions surrounded by a plurality of bonds, and a hollow projection is formed in each of the regions surrounded by the bonds. Each continuously raised portion had a continuous projection continuously extending the whole length of the topsheet in the lateral direction, the continuous projection being composed of a plurality of sub-projections connected in series.

The bond (projection) formation pattern in the topsheet used in Example 3 was similar to the pattern in the topsheet 2 (see FIG. 4). Specifically, the projections of the topsheet of Example 3 included no continuous projections but discrete hollow projections each formed in a region surrounded by a plurality of bonds (a bond-surrounded region). These projections were discretely and regularly arranged over the entire area of the skin facing side of the topsheet. The bonds surrounding every single projection were spaced around the projection.

### Comparative Examples 1 and 2

A commercially available disposable diaper (GOO.N, produced in 2015 by Oji Paper Co., Ltd.) was used as a sample of Comparative Example 1.

Another commercially available disposable diaper (Genki!, produced in 2015 by Oji Paper Co., Ltd.) was used as a sample of Comparative Example 2.

### Evaluation Test:

Each of the topsheets of Examples and Comparative Examples was examined by determining shear strength by the method described supra and determining MMD (mean deviation of coefficient of friction) and micro-compression strength by the methods described below.

MMD is a measure of surface roughness of the skin facing side of the topsheet under examination. A smaller MMD value indicates lower friction with the wearer's skin, meaning a higher rating. A smaller micro-compression strength indicates that the topsheet is softer. Accordingly, the softness of the topsheet under examination, which will lead to the wearer comfort of an absorbent article using the topsheet, can be evaluated from the MMD and micro-compression strength.

### Method for Determining MMD:

The MMD of the skin facing side (textured side) of the topsheet under evaluation was determined in the longitudinal direction of the disposal diaper (corresponding to the front-to-back direction of a wearer) using a commercially available instrument (KES-FB 4 surface tester, from Kato Tech Co., Ltd.) under conditions of SENS of 2×5 and a load of 50 gf/cm². The MMD value is a measure of surface roughness of an object under examination. The smaller the MMD, the lower the friction with the wearer's skin, indicating a higher rating.

### Method for Determining Micro-Compression Strength:

The micro-compression strength of the topsheet under examination was determined using a commercially available instrument (KES-FB3 compression tester, from Kato Tech Co., Ltd.) under conditions of a rate of compression of 0.02 mm/s, a compressive load of 10 gf/cm², and an assessment length of 0.8 mm. The smaller the micro-compression strength, the softer the object under examination.

### Method for Evaluating Lift-Off of Topsheet:

A specimen quadrilateral in plan view was cut out of the topsheet under evaluation, and its textured side was used as the surface to be evaluated. The specimen was placed on a flat surface of a plate with its textured side up, and the four sides of the specimen were fixed to the plate with gummed tape so as to bring the entire area of the non-textured side of the specimen into intimate contact with the plate. In this state, there was no separation (lift-off) of the specimen from the plate. A friction disc wrapped in sponge (Moltopren MF-30) was placed on the specimen to give a load of 240 g and given 15 cycles of turns, each cycle consisting of three clockwise turns followed by three counterclockwise turns, taking 3 seconds for each turn. After 15 cycles of turns, the degree of lift-off of the specimen from the plate was observed by the naked eye. A gap between the plate and even a part of the specimen as observed shall mean occurrence of lift-off.

Another specimen cut out of the same topsheet was tested in the same manner to see whether lift-off occurred.

The above test was repeated a plurality of times, and the rate of lift-off occurrence was calculated by the formula below. The topsheet was rated “A”, a highest rating, when the lift-off occurrence rate was 0% (no lift-off of the topsheet observed); B for a lift-off occurrence rate less than 5%: C for 5% to 20%; D for 21% to 50%; and E for 51% or less.

Lift-off occurrence rate=(area of lift-off part of topsheet/total area of topsheet per diaper)×100.

TABLE 1

| | | Example | | | Comparative Example | |
|---|---|---|---|---|---|---|
| | | 1 | 2 | 3 | 1 | 2 |
| Topsheet | Projection Height (mm) | 0.90 | 0.63 | 0.81 | 0.39 | 0.46 |
| | Number of Projections per | 24 | 30 | 28 | 28 | 6 |
| | Rectangular Region | | | | | |
| | Bond Area Ratio (%) | 21.3 | 26.7 | 24.0 | 26.9 | 10.5 |
| | Shear Strength (N) | 27.7 | 26.0 | 25.4 | 20.3 | 12.3 |
| Results of | MMD | 0.0070 | 0.0087 | 0.0083 | 0.0078 | 0.0089 |
| Evaluation | Micro-compression Strength | 4.2 | 5.5 | 4.5 | 3.8 | 3.5 |
| | (gf/cm) | | | | | |
| | Lift-off of Topsheet | A | B | B | C | D |

As is shown in Table 1, the topsheet of each of the disposable diapers of Examples satisfies all the conditions (1) to (4) and is therefore rated B or higher, i.e., their lift-off occurrence rate was less than 5%. It is thus seen that these topsheets are less likely to lift during use. Furthermore, the topsheet of each Example has sufficiently small values of MMD and micro-compression strength. This indicates that the topsheet is soft and has reduced friction with the wearer's skin, providing excellent wearer comfort. In particular, the diaper of Example 1 shows better results in evaluation than those of Examples 2 and 3. This proves that the previously stated narrower ranges of the conditions (1) to (4), in which the values adopted in Example 1 fall, are particularly preferred.

The diaper of Comparative Example 1 satisfies the conditions (2) and (3) but not the conditions (1) and (4). As a result, it was rated poorer in terms of resistance to lift-off of the topsheet than those of Examples. The diaper of Comparative Example 2 satisfies none of the conditions (1) to (4) and was therefore rated still poorer than that of Comparative Example 1.

### INDUSTRIAL APPLICABILITY

The invention provides an absorbent article of which the topsheet adapted to contact the skin of a wearer is soft and less likely to lift off during wear.

1. An absorbent article comprising an absorbent member and a topsheet on the skin facing side of the absorbent member and having a longitudinal direction corresponding to the front-to-back direction of a wearer and a lateral direction perpendicular to the longitudinal direction,
the topsheet comprising a stack of a first sheet and a second sheet bonded to each other at a plurality of bonds,
the first sheet having a plurality of projections toward the skin of a wearer resulting from the first sheet protruding away from the second sheet at portions other than the bonds, and,
the topsheet satisfying the following conditions (1) to (4):
(1) the projections having a height of 0.5 to 10 mm,
(2) the bonds having a density of 10 to 30 bonds per a randomly chosen rectangular region of the topsheet measuring 10 mm in length and 15 mm in width in plan view,
(3) the bonds in the rectangular region having a total area ratio of 12% to 35% relative to the area of the rectangular region, and
(4) the topsheet having a shear strength of 21 to 70 N.

2. The absorbent article according to claim 1, wherein the plurality of bonds are arranged in a discrete pattern in plan view.

3. The absorbent article according to claim 2, wherein the plurality of bonds are arranged discretely in a regular pattern over the whole area of the topsheet such that the plurality of projections are each surrounded by at least two of the plurality of bonds spaced from each other around each projection.

4. The absorbent article according to claim 2, wherein the projections are each surrounded by more than two of the plurality of bonds and have their respective tops positioned at substantially the center of the region surrounded by the more than two bonds.

5. The absorbent article according to claim 1, wherein the projections have a height of 0.6 to 5 mm.

6. The absorbent article according to claim 1, wherein the density of the bonds is 15 to 28 bonds per rectangular region.

7. The absorbent article according to claim 1, wherein the bonds in the rectangular region has a total area ratio of 15% to 30% relative to the area of the rectangular region.

8. The absorbent article according to claim 1, wherein the shear strength is 23 to 40 N.

9. The absorbent article according to claim 1, wherein the topsheet comprises fusible conjugate fibers made up of more than one resin and has a large number of intersections of the fusible conjugate fibers, the fusible conjugate fibers being fusion-bonded to each other at at least part of their intersections.

10. The absorbent article according to claim 1, wherein the plurality of bonds comprise a plurality of first bonds having a square plan-view shape and a plurality of second bonds having a rectangular plan-view shape.

11. The absorbent article according to claim 10, wherein each second bond is larger in area than the first bond and longer in the longitudinal direction than in the lateral direction.

12. The absorbent article according to claim 10, wherein the plurality of first bonds are spaced in the lateral direction in straight rows, and the plurality of second bonds are spaced in the lateral direction in straight rows, the rows of the first bonds and the rows of the second bonds alternating in the longitudinal direction such that the first bonds and second bonds in adjacent rows do not align in the longitudinal direction.

13. The absorbent article according to claim 10, wherein the plurality of projections comprise a first projection having a circular plan-view shape and a second projection having an elliptic plan-view shape of which the longer axis is coincident with the lateral direction, and the second projection is larger in area than the first projection.

14. The absorbent article according to claim 13, wherein the second projection is higher than the first projection and the highest of all the plurality of projections.

15. The absorbent article according to claim 13,
wherein a ratio of the height of the second projection to that of the first projection, second projection/first projection, is 1.1 to 5.0.

16. The absorbent article according to claim 13, wherein the first projection comprises a plurality of first protrusions, and the second projection comprises a plurality of second protrusions, the plurality of first projections being spaced in the lateral direction in straight rows, the plurality of second projections being spaced in the lateral direction in straight rows, and the rows of first projections and the rows of second projections alternating in the longitudinal direction in such a pattern that one pair of adjacent first projections and one second projection are aligned in the longitudinal direction.

17. The absorbent article according to claim 13, wherein the first projection is surrounded by a total of four bonds consisting of two first bonds aligned in the longitudinal direction and two second bonds aligned in the lateral direction, and the second projection is surrounded by six first bonds.

18. The absorbent article according to claim 1, wherein the highest projection of the plurality of projections has a length in the longitudinal direction and a length in the lateral direction that is longer than the length in the longitudinal direction, both measured along the outer surface of the projection.

19. The absorbent article according to claim 18, wherein the highest projection has a ratio of the length in the lateral direction to the length in the longitudinal direction, lateral direction/longitudinal direction, of 1.1 to 5.0.

20. The absorbent article according to claim 18, wherein the highest projection has a length of 1.5 to 10 mm measured along the outer surface thereof in the longitudinal direction.

21. The absorbent article according to claim 18, wherein the highest projection has a length of 2.0 to 10 mm measured along the outer surface thereof in the lateral direction.

22. The absorbent article according to claim 1, wherein the absorbent member or an absorbent core as a main component of the absorbent core has a single layer structure and being hourglass-shaped with its longitudinal middle portion narrowed in plan view.
